(19) United States
(12) Patent Application Publication
(10) Pub. No.: US 2025/0010023 A1
(43) Pub. Date: Jan. 9, 2025

(54) CATHETER ASSEMBLY

(71) Applicant: ConvaTec Limited, Flintshire (GB)

(72) Inventor: Oliver Pfleger, Spital (GB)

(21) Appl. No.: 18/891,444

(22) Filed: Sep. 20, 2024

Related U.S. Application Data
(63) Continuation of application No. PCT/GB2023/050662, filed on Mar. 20, 2023.

(30) Foreign Application Priority Data
Mar. 21, 2022 (GB) GB2203900.2

(51) Int. Cl. A61M 25/00

(52) U.S. Cl. CPC A61M 25/0017; A61M 25/002; A61M 2205/0216; A61M 2205/582; A61M 2205/583; A61M 2207/10; A61M 2209/06; A61M 2210/1089

(57) ABSTRACT

A packaged catheter assembly that has a catheter arranged within a sterile sealed pouch. The pouch has an openable top and a resealing arrangement proximal the openable top for resealing the pouch after use. A portion of the catheter is arranged in the sealed pouch so as to obstruct the resealing arrangement.

### CROSS REFERENCE TO RELATED DISCLOSURES

The present disclosure is a Continuation of International Application No. PCT/GB2023/050662 filed Mar. 20, 2023, and claims the benefit of GB 2203900.2 filed Mar. 21, 2022, the contents of which being incorporated herein by reference in entirety.

### TECHNICAL FIELD OF THE INVENTION

The present invention relates at its broadest to medical devices in general, but primarily to catheter assemblies. In particular the invention concerns packaged urinary catheter assemblies, and most particularly, but not exclusively packaged intermittent male urinary catheter assemblies.

### BACKGROUND TO THE INVENTION

Many people suffer from urinary problems that can make it difficult to pass urine. Intermittent catheter assemblies can provide a convenient and portable solution to this problem as a user is able to self-catheterise to relieve themselves as required. This reduces the effect of their urinary condition on their life and allows them to enjoy a relatively normal lifestyle.

To reduce the risk of damage to the urinary tract and improve comfort while using the catheter, it is important that catheters are activated before use for example through wetting of the catheter by a wetting fluid. However, it is desirable to avoid providing too much wetting fluid, as excess fluid can leak out of the package in which it is provided, and of course, simply to reduce material cost. Various approaches exist to avoiding excess free-fluid in the package for a catheter, such as providing the catheter in a vapour rather than having a separate container of liquid that is opened to wet the catheter. However, it would be desirable to improve on the simple approach of wetting the catheter by opening a container of fluid into a pouch which houses the catheter, then drawing the catheter through the pool of fluid to wet it.

Where a catheter is packaged in a pouch, it has been proposed, for example in WO2021/214478A1 to provide a resealing arrangement, which can seal the pouch after use. It is noted that configuring the packaging such that it is sealable following opening may be beneficial in situations wherein liquid might be present (e.g. urine or wetting fluid for catheter), and/or where a user wishes to keep the contents of the packaging discreet, e.g. on exit from a toilet cubicle before disposal. As such, the pouch of WO2021/214478A1 is openable by tearing off a tear-away region at the top of the pouch, but has a strip of hook fasteners which is secured (e.g. adhesively) to the interior surface of the respective side of the packaging, so as to form a double hook fastener arrangement for resealing the open upper end of the packaging, in use. There is a downside to this arrangement in that the double hook fasteners can tend to close before use, such that the user has to first tear off the tear away region, then pull open the double-hook fasteners to access the contents of the pouch. This can be troublesome, especially for those with low manual dexterity—a fairly common trait amongst intermittent catheter users.

Yet another problem with packaged catheter assemblies as proposed in the prior art concerns correct use of the catheter. In particular, where a catheter is packaged with a container of wetting fluid, the user may be tempted to open the pouch that houses the catheter and the wetting fluid, then reach into the pouch to burst the container of wetting fluid. This risks contaminating, or dirtying the catheter and wetting the user's hand unnecessarily. The correct approach is to open (e.g. burst) the container from the outside, by squeezing it through the material of the pouch, ideally prior to opening the pouch. It would be beneficial to promote this approach.

It is an object of embodiments of the present invention to at least partially overcome or alleviate the above problems and/or to provide an improved catheter assembly.

### SUMMARY OF THE INVENTION

In broad terms, aspects of the invention concern a medical device. The medical device may be a packaged medical device. The medical device may be a catheter assembly. The packaged medical device may be a packaged catheter assembly comprising a catheter arranged within a pouch. The pouch may further comprise a container of wetting fluid. The pouch may have a pair of major edges or lateral edges (i.e. the major edges may or may not be lateral edges and the lateral edges may or may not be major edges). The pouch may have an openable edge, which may be the top. The pouch may have a base opposing the top. The base may be contoured. The base may protrude downwards beyond the point where it meets the lateral edges. The catheter may be arranged within a sterile sealed pouch. The pouch may have a resealing arrangement for resealing the pouch after use. The resealing arrangement may be proximal the openable top. A portion of the medical device may be arranged in the sealed pouch so as to obstruct the resealing arrangement. A portion of the catheter may be arranged in the sealed pouch so as to obstruct the resealing arrangement. The pouch may have first and second walls joined around the periphery thereof. The join around the periphery may define the pair of major or lateral edges, a base and a top edge. The openable top when opened may have a mouth at its upper end to allow the catheter to be withdrawn. A lateral join may be provided, which may extend inwardly from a major edge and may obscure visibility of and/or restrict access to the container of wetting fluid. The pouch may be configured to be opened, for example cut open, peeled open or most preferably torn open. It may be configured to be opened at an upper edge of the pouch to form an open upper end of the pouch in use. The pouch may have a tear-away region, which may be defined proximal to an upper edge of the pouch. The tear away region may comprise a tear line between a first end and a second end of the tear-away region, defining a line along which the pouch may be torn to at least partially separate the tear-away region from the remainder of the pouch to form an open upper end of the pouch, in use. The tear-away region may comprise a first interaction region defining a point of contact for a user to grasp the tear away region. The remainder of the pouch may have a corresponding second interaction region defining a point of contact for a user to grasp the remainder of the pouch. The first and second interaction regions may comprise one or more visual and/or tactile indicators, which may be provided at the same major edge and may be separated from each other by the tear line. In use a user may grasp the first interaction region with one hand and grasp the other (second) interaction region with another hand to tear open the pouch. The container of wetting fluid may be arranged at the second interaction region. Squeezing the pouch at the second interaction region may rupture the container of wetting fluid.

According to a first aspect of the invention there is provided a packaged catheter assembly comprising a catheter and a container of wetting fluid arranged within a pouch; the pouch having a pair of lateral edges, a top and an opposing base; wherein the base protrudes downwards beyond the point where it meets the lateral edges.

Such a pouch is advantageous in two respects. Firstly, having a protruding base may make the pouch easier to pocket (i.e. to introduce into a pocket, such as a trouser-pocket). This is because the effect of the protrusion may be to render the base narrower at its end, allowing it to be inserted more easily into the pocket. Secondly, when the pouch is held upright, after opening (e.g. rupturing) the container of wetting fluid, the fluid will preferentially accumulate in the protruding base, and on account of the protrusion, a deeper pool of fluid may be provided than would be the case if the base ran straight between the point where it meets the lateral edges. As such, less fluid may be required to provide a sufficient depth to adequately wet the catheter.

The base may be contoured. The term “contoured” used herein, is intended to define a shape other than a straight line between the point where the base meets the lateral edges.

The base may be rounded, for example, it may be part-circular. A rounded base is particularly beneficial in two further respects. Firstly, where the exterior of the base is rounded, it can be more smoothly pocketed, being less likely to catch on the pocket. Secondly, where the interior of the base is rounded, when, after use, the used catheter is re-introduced into the pouch prior to disposal, as the tip of the catheter abuts the rounded base, it will follow the contour of the base and curl up within the pouch.

The base may be semi-circular. Whilst other rounded contours are contemplated, a semi-circular base (i.e. one where the outline of the base is the outline of half a circle) will be particularly effective in terms of the benefits outlined above.

The base may be sloped. A sloped base is an alternative to a rounded base and may serve the same purpose. The base may be sloped on two sides, for example, the base may be “triangular”, with two sloped sides extending from the point where the base meets the lateral edges and meeting at a corner, and a third side being defined as the line between the points where the base meets the lateral edges. The base may be an isosceles triangle, in which case, the two sloped sides may be shorter than the line between the points where the base meets the lateral edges. The base may be an equilateral triangle, in which case the two sloped sides may be equal in length to the line between the points where the base meets the lateral edges.

Of course, the base could alternatively combine a combination of rounded and sloped features: for example, the base could have two sloped sides, and as such be generally triangular, but could be rounded where the two sloped sides meet, rather than having an abrupt corner. Similarly, the base could have two sloped sides, and as such be generally triangular, but could be rounded where the one or more of the sloped sides meet the lateral edges, rather than having an abrupt corner. Still further, the base could have one sloped side and one rounded side, for example, it could comprise a segment of a circle, such as a quarter-circle, extending from one lateral edge, which meets a sloped side, which extends to the other lateral edge.

The interior of the base may be shaped as defined above. The advantages in terms of wetting the catheter with less fluid and more easily re-introducing the catheter to the pouch after catheterisation stem primarily from the shape of the interior of the base of the pouch. As such, they could be achieved even if the exterior shape of the base was different, for example if a contoured join (e.g. weld) defined the shape of the interior of the base, but a separate straight join (e.g. weld) was provided beneath the contoured join to define the shape of the exterior of the base.

The exterior of the base may be shaped as defined above. The advantages in terms of easily pocketing the catheter stem primarily from the shape of the exterior of the base. As such, they could be achieved even if the interior shape of the base was different, for example, if a contoured weld defined the shape of the exterior of the base, but a separate straight join was provided above the contoured join to define the shape off the interior of the base.

Of course, it is preferred that the shape of both the interior and exterior of the base are as defined. This not only achieves more benefits, but can be easier to produce, as a single join (e.g. weld) having the same contour can define both the interior and the exterior shape of the base.

The pouch may be formed of two walls of flexible sheet material. The two walls may be joined at their periphery. For example, they may be joined by a weld, or adhesive. The peripheral seal may comprise any one or more of: a weld; mechanical seal; heat seal; pressure seal; adhesive; solvent bond; ultraviolet bond; ultrasonic weld; laser weld; impulse weld; or friction weld. They may also be joined by a fold, that is to say, one sheet of flexible material may be folded to provide two opposing walls, which may then be joined to define the remainder of the periphery of the pouch.

The pouch may comprise a flexible plastics material. For example, polypropylene (PP), polyethylene terephthalate (PET), low density polyethylene (LDPE), metalized polyester (MET PET) or orientated polypropylene (OPP).

The pouch may be formed from an opaque material. The pouch may have a textured or printed appearance and/or feel, such as a matte finish. The pouch may be a different colour from the other components of the packaged catheter assembly, such as any one or more of: the container of wetting fluid and the catheter. Thus, these components are easily identifiable in the pouch and the catheter assembly is more discrete as the appearance and feel of the pouch means it is less likely to be identified as a medical device.

The container of wetting fluid may be a rupturable sachet. It is very convenient to provide a rupturable sachet as the container of wetting fluid. The wetting fluid may be water and the catheter may have a hydrophilic surface. Water interacts with the hydrophilic surface of the catheter to render it lubricious. In other embodiments, other wetting fluids may be used and they may be polar (e.g. water-based) or non-polar (e.g. oil-based).

The container of wetting fluid may be configured to release the wetting fluid directly into the pouch. As such, the fluid may flow freely to the protruding base of the pouch. The container of wetting fluid may be configured to release wetting fluid when activated by a user. The fluid reservoir may be activated by a user by any suitable means, for example applying a compressive force to the fluid reservoir, in particular squeezing it through the pouch.

As outlined above, the top of the pouch may be openable. This is particularly preferred in this aspect of the invention, where fluid flows freely and accumulates at the protruding base, because the top is furthest from the base, so leakage is less likely than if a side of the pouch is openable instead.

The container of wetting fluid may contain at least 2, 3, 4, 5, 6, 7, 8, 9 or 10 ml of wetting fluid, most preferably at least 4 ml of fluid. The container of wetting fluid may contain no more than 20, 15, 12, 11, 10, 9, 8, 7, 6 or 5 ml of fluid, most preferably no more than 10 ml; for example between 4 and 8 ml, such as about 6 ml. This is less fluid than would normally be required to wet a catheter, at least a male catheter, in a packaged catheter assembly of this type and such a small amount may be adequate on account of the beneficial wetting achieved by the protruding base.

The catheter may be provided with a sleeve, but is preferably sleeveless. A sleeveless catheter is more easily wetted than one with a sleeve surrounding the catheter tube. If a sleeve is provided, the packaged catheter assembly (in particular the packaged catheter assembly in a sterile sealed state) preferably contains the sleeve in a furled configuration, or the sleeve may be short, i.e. not covering the full length of the catheter tube, but rather exposing part of it. As such, the majority of the catheter tube may be exposed despite the provision of a sleeve. For example, at least 40%, 50%, 60%, 70%, 80%, 90% or 95% of the catheter tube may be exposed. In another alternative, the sleeve may be permeable.

The catheter may be a urinary catheter. The catheter may be a male urinary catheter (i.e. a urinary catheter for men). Alternatively, it may be a female urinary catheter (i.e. a urinary catheter for women). The catheter may be an intermittent catheter. In one embodiment, the catheter is an intermittent male urinary catheter. Thus, the features of the present invention allow intermittent male urinary catheters to be adequately wetted prior to use which can be more difficult than for other types of catheter which are generally shorter. The catheter may for example be at least 20 cm, at least 25 cm, at least 30 cm, or at least 35 cm long. It may be no more than 50 cm, no more than 45 cm, or no more than 40 cm long, for example between 35 and 40 cm long, such as 37 cm long.

The packaged catheter assembly may be an open catheter assembly (as opposed to a closed catheter assembly) i.e. one in which the catheter tube is not connected to a urine bag, but rather is used to direct urine directly to a lavatory or the like.

The catheter may be arranged within a sterile sealed pouch; the pouch having an openable edge (e.g. an openable top) and a resealing arrangement (which may be proximal the openable top) for resealing the pouch after use; wherein a portion of the catheter is arranged in the sealed pouch so as to obstruct the resealing arrangement.

Accordingly, in a second aspect of the invention, there is provided a packaged catheter assembly comprising a catheter arranged within a sterile sealed pouch; the pouch having an openable edge (e.g. an openable top) and a resealing arrangement (which may be proximal the openable edge/top) for resealing the pouch after use; wherein a portion of the catheter is arranged in the sealed pouch so as to obstruct the resealing arrangement.

This arrangement, with a portion of the catheter obstructing the resealing arrangement, is advantageous in terms of preventing the resealing arrangement closing before the catheter is used. As such, the user will not have to open the resealing arrangement after opening the openable edge (breaking the sterile seal). This of course is beneficial especially for those with low manual dexterity.

In this specification, the expression “catheter” when used alone as a noun, as opposed to as a descriptor (e.g. catheter tube or catheter assembly) is intended to be read broadly as encompassing both a catheter tube alone (that being the simplest form of catheter and the fundamental element of a catheter) and a catheter assembly, a catheter assembly comprising both a catheter tube and at least one other element, such as a funnel, gripping element, sleeve, fluid collection bag and so forth. As such, the portion of the catheter which obstructs the resealing element may be the catheter tube itself, or where the catheter is a catheter assembly, it may be another element of the assembly.

The resealing arrangement may comprise opposing resealing elements, for example opposing strips, which engage to reseal the pouch. The portion of the catheter may be arranged between the opposing resealing elements (e.g. strips), to obstruct the resealing arrangement. The portion of the catheter may alternatively be arranged adjacent to the opposing resealing elements to obstruct the resealing arrangement. This can be effective by holding apart walls of the pouch that are attached to the opposing resealing elements (e.g. strips), so that the resealing elements cannot meet. That is to say, although the portion of the catheter is only physically between the walls adjacent to the resealing arrangement, an air gap will be produced between the opposing resealing elements. Obviously the air gap will only be maintained in the region close to the portion of the catheter and further away it will be possible to push opposing walls together, hence it is necessary that the portion of the catheter is at least adjacent the opposing resealing elements, if not physically between them.

The opposing resealing elements may comprise complementary attachment features on their opposing surfaces which interlock to reseal. For example, the opposing resealing elements may be strips of hook and loop (as commonly known by the Velcro™ brand), hook and hook, or “mushroom” fastening elements, alternatively, the opposing resealing strips could be a tongue and groove arrangement (as commonly known by the Ziploc™ brand). Each of these resealing arrangements should be familiar to and therefore well understood by users, and each would suffer the problem of potentially sealing before use, without a portion of the catheter being arranged to obstruct the resealing arrangement.

Alternatively, it is conceivable that the resealing arrangement may rely on a single resealing element, such as a single resealing strip. For example, the resealing element could be a strip of adhesive arranged adjacent the openable edge on one wall in order to reseal it by engagement with an opposing wall. In that case, it is preferred that the portion of the catheter is arranged between the strip of adhesive and the region of the opposing wall that opposes the strip of adhesive. The portion of the catheter may alternatively be arranged adjacent to the strip of adhesive to obstruct the resealing arrangement. This can be effective as outlined above because although the portion of the catheter is only physically between the walls adjacent to the adhesive, holding those apart, an air gap will be produced between adhesive and the wall opposite.

The portion of the catheter may obstruct the resealing arrangement such that it is not fully closed before use. That is to say, it may obstruct the resealing arrangement such that it is at least partially unsealed before use. The portion of the catheter may obstruct the resealing arrangement such that it is at least 10%, 20%, 30%, 40%, 50%, 60%, 70%, 80% or 90% unsealed before use. Obviously the greater the proportion of the resealing arrangement that is unsealed before use, the easier it is to access the contents. However, having a small proportion sealed before use by the resealing arrangement may not be a major issue. For example, where the pouch is formed from two opposing walls of flexible sheet material joined at their periphery, if the resealing arrangement is sealed before use near to the periphery, it will make the opening at the mouth smaller, but it will still be easier to access the contents than if the resealing arrangement was fully closed. It is preferred that a portion of the catheter is arranged in register with the resealing arrangement. When a portion of the catheter is in register with the resealing arrangement (e.g. physically between opposing resealing elements, or physically between an adhesive and the wall opposite), even if a greater part of the resealing arrangement is sealed before use, it may not be a problem, because the portion that is in register can be grasped to aid in opening the resealing arrangement more fully, or indeed, may open automatically as the catheter is pulled out.

The portion of the catheter may extend across at least 10%, 20%, 30%, 40%, 50%, 60%, 70%, 80% or 90% of the height of the resealing arrangement, for example extending across 95% or 100% of the height of the resealing arrangement.

The catheter is preferably formed of a resilient material. It is preferably biased towards a straighter shape. It is preferably urged against the base of the pouch and towards the top of the pouch.

Preferably the catheter extends over at least 70%, 80%, 90% or 95% of the height of the interior of the pouch, for example namely about 95% of the height of the interior of the pouch. Preferably the catheter extends over at least 70%, 80%, 90% or 95% of the distance from the base of the interior of the pouch to the top of the resealing arrangement, for example over about 95% of the distance from the base of the pouch to the top of the resealing arrangement.

The portion of the catheter may extend into a region between the openable edge and the resealing arrangement. The pouch may have a construction comprising a “body”, in which the majority of the catheter assembly, and optionally a container of wetting fluid is located, a resealing arrangement and a region between the openable edge and the resealing arrangement, and, as such, with the portion of the catheter (which obstructs closure of the resealing arrangement before use) extending all the way to the region between the resealing arrangement and the openable edge, it can be especially easy for a user with low manual dexterity to gain access to the catheter. This is because after opening the openable edge, the catheter assembly can be grasped in the region between the resealing arrangement and the mouth and pulled out.

The portion of the catheter that is arranged to obstruct the resealing arrangement may be the distal end of the catheter, for example it may be a funnel. A funnel will not be introduced into a user's urethra in-use, so (whilst damage by the resealing arrangement is unlikely) it is less of a concern if the funnel were to be at all damaged, e.g. scratched by the resealing arrangement. Also, a funnel is intended to be held by a user in-use, so it is beneficial for it to be presented to the user, at the mouth of the pouch (where the resealing arrangement is preferably arranged) rather than presenting a portion of the catheter that is not intended to be touched, e.g. the catheter tube itself.

The portion of the catheter that is arranged to obstruct the resealing arrangement may be a gripping element. Again, arranging a gripping element in the resealing arrangement is beneficial in that any slight scratches to the gripping element would not be an issue and in that it involves presenting to the user an element that it intended to be touched. The gripping element may be a handling tube extending around part of the catheter tube and may be moveable along the length of the catheter tube.

It is conceived that the catheter could be a closed-catheter assembly, with a catheter tube attached to a fluid collection bag, in that case, the fluid collection bag may be arranged to obstruct the resealing arrangement. The benefits, of course, are the same.

As outlined above, the pouch may have a tear-away region. The tear away region may be at the openable edge of the pouch. The tear away region may open the openable edge and thereby break the sterile seal. The tear away region may of course have any of the features outlined in relation to the broad aspect of the invention.

Preferably, (a) a lateral join is provided, extending inwardly from a major edge, obscuring visibility of and restricting access to the container of wetting fluid; and/or (b) the pouch has a tear-away region defined proximal to an upper edge of the pouch, the tear away region comprising a tear line between a first end and a second end of the tear-away region defining a line along which the pouch may be torn to at least partially separate the tear-away region from the remainder of the pouch to form an open upper end of the pouch, in use; the tear-away region comprising a first interaction region defining a point of contact for a user to grasp the tear away region; and the remainder of the pouch having a corresponding second interaction region defining a point of contact for a user to grasp the remainder of the pouch; the first and second interaction regions comprising one or more visual and/or tactile indicators, being provided at the same major edge and being separated from each other by the tear line; (preferably whereby in use a user may grasp first interaction region with one hand and grasp the other interaction region with another hand to tear open the pouch); and the container of wetting fluid being arranged at the second interaction region (preferably such that squeezing the pouch at the second interaction region ruptures the container of wetting fluid).

Thus, in a third aspect of the invention, there is provided a packaged catheter assembly comprising a catheter and a container of wetting fluid arranged within a pouch; the pouch comprising first and second walls joined around the periphery thereof to define a pair of major edges, a base and a top edge; the pouch having an openable top, which when opened has a mouth at its upper end to allow the catheter to be withdrawn; wherein a lateral join is provided, extending inwardly from a major edge, obscuring visibility of and restricting access to the container of wetting fluid.

The advantage of this arrangement is that if the container cannot be seen from the top, it is unlikely that a user will remove it. Consequently, it is hoped and expected that it is more likely that a user will open the container of liquid in-situ. The benefit of this is that spillage of the wetting fluid is less likely and therefore more of the fluid will be present in the pouch to fulfil its function of wetting the catheter (and users are less likely to wet their hands, which is undesirable). The lateral join may also act to block fluid from spurting out of the top of the pouch, if it is ruptured after opening the pouch and may reduce the chances of the container itself being accidentally squeezed out of the pouch instead of rupturing.

The lateral join may comprise a weld. A weld (in particular a plastic weld between opposing walls of plastics material) is a convenient way to form the join. As outlined above, the first and second walls may be joined around the periphery and that join may be a weld, as such, the lateral join can be formed simultaneously with the formation of the peripheral join.

The lateral join may be provided at or proximate to the mouth of the pouch. The length of the pouch when open may be defined as the distance between the mouth of the pouch and its base and the lateral join may be provided no more than 30%, no more than 20%, no more than 10% or no more than 5% of the length of the pouch from the mouth.

The lateral join may define the length of the opening. The length of the opening may be the distance from the tip of the lateral join (distal the major edge from which it extends) to the opposing major edge. The length of the opening may be shorter than the distance between the major edges, which may define the length of the mouth. As such, the length of the opening may be shorter than the length of the mouth.

The lateral join may be at least 1 cm, at least 1.5 cm or at least 2 cm long (measured from the major edge from which the lateral join extends to its tip). This should be sufficient to adequately restrict visibility of, and access to, the container of wetting fluid. The lateral join may be no more than 6 cm, 4 cm, 3 cm or 2 cm long. This balances the amount to which the join restricts the size of the opening against the degree to which it obscures the container.

The lateral join may extend at an angle of at least 45 degrees with respect to the major edge from which it extends, preferably at least 70 degrees and is preferably perpendicular (i.e. substantially perpendicular) to the major edge from which it extends.

The container of wetting fluid may be arranged adjacent the major edge from which the lateral weld extends. The container of wetting fluid may be arranged between the lateral join and the base of the pouch.

The container of wetting fluid may have a polygonal outline. The container of wetting fluid may have a length, a width and a depth. The length (the longest dimension of the length, width and depth) may be aligned with the major edge from which the lateral join extends. For example, the length may be parallel (i.e. substantially parallel) with the major edge. The length of the wetting container may be at least 3 cm, at least 4 cm, at least 5 cm, at least 6 cm or at least 7 cm. The length may be no more than 15 cm, no more than 10 cm or no more than 8 cm, for example between 7 and 8 cm, such as 78 mm. The width of the container of wetting fluid may extend across the pouch, for example, it may extend from the major edge adjacent to which it is arranged, towards an opposing major edge. The container of wetting fluid may have a rectangular outline. The width of the container may be less than twice the length of the lateral join, less than 1.5 times the length of the lateral join, or (preferably) less than or equal to the length of the lateral join, for example less than 95%, 90%, 80% or 70% of the length of the lateral join. The width of the container may be at least 1 cm, at least 1.5 cm or at least 2 cm. The width may be no more than 6 cm, 4 cm, 3 cm or 2 cm long.

Preferably the pouch has a break-away region e.g. a peel-away or more preferably a tear-away region defined proximal to an upper edge of the pouch, the break away (e.g. tear away) region comprising a break line (e.g. tear line) between a first end and a second end of the break away (e.g. tear-away) region defining a line along which the pouch may be opened (e.g. torn to at least partially separate the tear-away region from the remainder of the pouch) to form an open upper end of the pouch, in use; the break away (e.g. tear-away) region comprising a first interaction region defining a point of contact for a user to grasp the break away (e.g. tear away) region; and the remainder of the pouch having a corresponding second interaction region defining a point of contact for a user to grasp the remainder of the pouch; the first and second interaction regions comprising one or more visual and/or tactile indicators, being provided at the same major edge and being separated from each other by the break line (e.g. tear line); (preferably whereby in use a user may grasp first interaction region with one hand and grasp the other interaction region with another hand to break (e.g. tear) open the pouch); and the container of wetting fluid being arranged at the second interaction region (preferably such that squeezing the pouch at the second interaction region ruptures the container of wetting fluid).

Thus, in one embodiment of the invention, there is provided a packaged catheter assembly comprising a catheter and a container of wetting fluid arranged within a pouch; the pouch comprising first and second walls joined around the periphery thereof to define a pair of major edges, a base and a top edge; the pouch having an openable top, which when opened has a mouth at its upper end to allow the catheter to be withdrawn; wherein a lateral join is provided, extending inwardly from a major edge, obscuring visibility of and restricting access to the container of wetting fluid; wherein the pouch has a break-away region defined proximal to an upper edge of the pouch, the break away region comprising a break line between a first end and a second end of the break-away region defining a line along which the pouch may be opened to form an open upper end of the pouch, in use; the break-away region comprising a first interaction region defining a point of contact for a user to grasp the break away region; and the remainder of the pouch having a corresponding second interaction region defining a point of contact for a user to grasp the remainder of the pouch; the first and second interaction regions comprising one or more visual and/or tactile indicators, being provided at the same major edge and being separated from each other by the break line; (preferably whereby in use a user may grasp first interaction region with one hand and grasp the other interaction region with another hand to break open (e.g. peel open) the pouch); and the container of wetting fluid being arranged at the second interaction region (preferably such that squeezing the pouch at the second interaction region ruptures the container of wetting fluid).

Thus, in one embodiment of the invention, there is provided a packaged catheter assembly comprising a catheter and a container of wetting fluid arranged within a pouch; the pouch comprising first and second walls joined around the periphery thereof to define a pair of major edges, a base and a top edge; the pouch having an openable top, which when opened has a mouth at its upper end to allow the catheter to be withdrawn; wherein a lateral join is provided, extending inwardly from a major edge, obscuring visibility of and restricting access to the container of wetting fluid; wherein the pouch has a tear-away region defined proximal to an upper edge of the pouch, the tear away region comprising a tear line between a first end and a second end of the tear-away region defining a line along which the pouch may be torn to at least partially separate the tear-away region from the remainder of the pouch to form an open upper end of the pouch, in use; the tear-away region comprising a first interaction region defining a point of contact for a user to grasp the tear away region; and the remainder of the pouch having a corresponding second interaction region defining a point of contact for a user to grasp the remainder of the pouch; the first and second interaction regions comprising one or more visual and/or tactile indicators, being provided at the same major edge and being separated from each other by the tear line; (preferably whereby in use a user may grasp first interaction region with one hand and grasp the other interaction region with another hand to tear open the pouch); and the container of wetting fluid being arranged at the second interaction region (preferably such that squeezing the pouch at the second interaction region ruptures the container of wetting fluid).

Correspondingly, in a fourth aspect of the invention, there is provided a packaged catheter assembly comprising a catheter and a container of wetting fluid arranged within a pouch; the pouch comprising first and second walls joined around the periphery thereof to define a pair of major edges, a base and a top edge; the pouch having an openable top, which when opened has a mouth at its upper end to allow the catheter to be withdrawn; wherein the pouch has a tear-away region defined proximal to an upper edge of the pouch, the tear away region comprising a tear line between a first end and a second end of the tear-away region defining a line along which the pouch may be torn to at least partially separate the tear-away region from the remainder of the pouch to form an open upper end of the pouch, in use; the tear-away region comprising a first interaction region defining a point of contact for a user to grasp the tear away region; and the remainder of the pouch having a corresponding second interaction region defining a point of contact for a user to grasp the remainder of the pouch; the first and second interaction regions comprising one or more visual and/or tactile indicators, being provided at the same major edge and being separated from each other by the tear line; (preferably whereby in use a user may grasp first interaction region with one hand and grasp the other interaction region with another hand to tear open the pouch); and the container of wetting fluid being arranged at the second interaction region (preferably such that squeezing the pouch at the second interaction region ruptures the container of wetting fluid).

The advantages of this aspect of the invention are also concerned with encouraging the opening of the wetting fluid before the pouch is opened, in order to reduce the prospect of spillage. In this aspect of the invention, the arrangement of an interaction region, which is intended to be grasped when opening the pouch, should result in the container of wetting fluid being opened when the user grasps the second interaction region, just prior to opening, if it has not already been opened. In preferred embodiments if the second interaction region is grasped tightly enough to tear the tear-away region, the container of wetting fluid automatically opens (e.g. it is a sachet, which will rupture under such force). Alternatively, at the very least, the user will notice the presence of the container of wetting fluid when grasping the second interaction region and will be encouraged to open it (typically by squeezing), so as to make it easier to hold the pouch and thus easier to open the pouch.

The second interaction region may overlap with the container of wetting fluid. For example, the second interaction region may extend from the major edge of the pouch towards an opposing major edge, and the container of wetting fluid may be located beneath the wall upon which the second interaction region is arranged and in register with the second interaction region. The second interaction region may be provided on both the first and the second walls and the container of wetting fluid may be located between the second interaction region on one wall and the second interaction region on the other wall.

The second interaction region may completely overlap the container of wetting fluid, i.e. the outline of the container of wetting fluid may be entirely within the outline of the second interaction region. As such, if pressure is applied to the entirety of the second interaction region, that pressure is sure to bear on the container of wetting fluid. The container of wetting fluid may completely overlap the second interaction region, i.e. the outline of the second interaction region may be entirely within the outline of the container of wetting fluid. As such, the second interaction region can be located on a particular area of the container of wetting fluid, e.g. a pressure point which will cause it to open; thus, pressure on the second interaction region can be certain to open the container of wetting fluid.

The second interaction region may exactly overlap with the container of wetting fluid, i.e. the outlines of the container of wetting fluid and the second interaction region may be the same and be exactly in register.

The second interaction region and the container of wetting fluid may alternatively be partially overlapping, i.e. the second interaction region may overlap only part of the container of wetting fluid and the container of wetting fluid may overlap only part of the second interaction region.

The second interaction region may comprise a rupture indicator, corresponding to a point which when squeezed will rupture the container of wetting fluid. For example, the rupture indicator may be a mark, e.g. a visible mark of a different colour to the remainder of the interaction region.

One or both of the visual or tactile indicators and especially the visual or tactile indicator of the second interaction region may be a pattern, for example grip-type patterns, such as a pattern of dots or stripes. The patterns may stand out from the remainder of the surface, for example they may be embossed or debossed.

The visual or tactile indicator of the first interaction region may be a gripping hole, e.g. a finger-hole. A gripping hole is especially useful for the first interaction region as in addition to indicating where to grasp the tear away region, it can help improve the user's grasp on that region. The gripping hole may provide a grippy surface or feature. The gripping hole may be any suitable size or shape, for example, rectangular, square, circular, elliptical. In one embodiment, the gripping hole is circular.

The packaged catheter assembly may further comprise a secondary join, provided inward of the periphery to maintain the location of the container of wetting solution. Alternatively, the location of the container of wetting solution could be maintained by being wedged into position (i.e. gripped by the two walls between which it is arranged). In yet another alternative, the location of the container of wetting solution could be maintained by arranging the contents of the pouch so as to hold the container of wetting solution in position; for example the catheter could be curled up so as to bear on the side of the container of wetting solution opposing the major edge that it is adjacent to, and thereby press it against the major edge.

Of course, the packaged catheter assemblies of any of the first to fourth aspects of the invention may comprise the features of other aspects of the invention, e.g. the packaged catheter assembly of the first aspect of the invention may comprise the features of the second, third and fourth aspects of the invention. It is particularly desirable to provide a packaged catheter assembly comprising both the features of the third and fourth aspects of the invention in light of the synergy between those aspects.

In addition, the packaged catheter assemblies of the first, second, third or fourth aspects may include any of the optional features of the first to fourth aspects without necessarily including all the features required of them. That is to say, an optional feature which happens to be set out following one particular aspect does not necessarily apply only to that aspect, so, for example, although details of preferred aspects of the container of fluid (such as it being a rupturable sachet, and certain preferred sizes) are set out in relation to the first aspect of the invention, they are equally applicable to the other aspects, and indeed they are particularly relevant to the third and fourth aspects of the invention which concern the encouragement of opening the container inside the sealed pouch, which is especially applicable where the container is a rupturable sachet.

According to a fifth aspect of the invention there is provided a method of forming a packaged catheter assembly; the method comprising providing a catheter and a container of wetting fluid; and arranging the catheter and container within a pouch; the pouch having a pair of lateral edges, a top and an opposing base; wherein the base protrudes downwards beyond the point where it meets the lateral edges.

The method of the fifth aspect of the invention may be a method of forming a packaged catheter assembly according to the first aspect of the invention and may of course include any optional feature outlined above.

According to a sixth aspect of the invention there is provided a method of forming a packaged catheter assembly comprising a catheter arranged within a sterile sealed pouch; method comprising forming a pouch having an openable top; providing a resealing arrangement (which may be proximal the openable top) for resealing the pouch after use; arranging a portion of the catheter so as to obstruct the resealing arrangement when the pouch is sealed with the sterile seal; and sealing and sterilising the pouch.

The method of the sixth aspect may be a method of forming a packaged catheter assembly according to the second aspect of the invention and may of course include any optional feature outlined above.

According to a seventh aspect of the invention, there is provided a method of forming a packaged catheter assembly comprising a catheter and a container of wetting fluid arranged within a pouch; the method comprising joining first and second walls around the periphery thereof to form a pouch and define a pair of major edges, a base and a top edge thereof; providing the pouch with an openable top, which when opened has a mouth at its upper end to allow the catheter to be withdrawn; and providing a lateral join, extending inwardly from a major edge, obscuring visibility of and restricting access to the container of wetting fluid.

The method of the seventh aspect may be a method of forming a packaged catheter assembly according to the third aspect of the invention and may of course include any optional feature outlined above.

According to an eighth aspect of the invention there is provided a method of forming a packaged catheter assembly comprising a catheter and a container of wetting fluid arranged within a pouch; the method comprising joining first and second walls around the periphery thereof to define a pair of major edges, a base and a top edge of a pouch; the pouch having an openable top, which when opened has a mouth at its upper end to allow the catheter to be withdrawn; providing the pouch with a tear-away region defined proximal to an upper edge of the pouch, the tear away region comprising a tear line between a first end and a second end of the tear-away region defining a line along which the pouch may be torn to at least partially separate the tear-away region from the remainder of the pouch to form an open upper end of the pouch, in use; providing, at the tear-away region, a first interaction region defining a point of contact for a user to grasp the tear away region; and providing on the remainder of the pouch a corresponding second interaction region defining a point of contact for a user to grasp the remainder of the pouch; the first and second interaction regions comprising one or more visual and/or tactile indicators, being provided at the same major edge and being separated from each other by the tear line; (preferably whereby in use a user may grasp first interaction region with one hand and grasp the other interaction region with another hand to tear open the pouch); and arranging the container of wetting fluid at the second interaction region (preferably such that squeezing the pouch at the second interaction region ruptures the container of wetting fluid).

The method of the eighth aspect may be a method of forming a packaged catheter assembly according to the fourth aspect of the invention. The method may further comprise the steps of the seventh aspect of the invention. Moreover, of course, the method of the eighth aspect of the invention may include any optional feature outlined above.

According to a ninth aspect of the invention, there is provided a method of wetting a catheter in a packaged catheter assembly comprising the catheter and a container of wetting fluid arranged within a pouch; the pouch having a pair of lateral edges, a top and an opposing base; wherein the base protrudes downwards beyond the point where it meets the lateral edges; the method comprising opening the container of wetting fluid, so that it forms a pool in the protruding base and pulling out the catheter through the pool of wetting fluid.

According to a tenth aspect of the invention, there is provided a method of disposing of a used catheter the method comprising introducing the used catheter to a pouch; the pouch having a pair of lateral edges, a top and an opposing base; wherein the base protrudes downwards beyond the point where it meets the lateral edges; the method comprising pushing the used catheter through the top to the base of the pouch, such that the an end of the catheter is directed by the protruding base so as to extend across the base of the pouch, then back up towards the top.

Of course, in both the ninth and tenth aspects of the invention the packaged catheter assembly may be according to the first aspect of the invention and may of course include any optional feature outlined above.

The method of the ninth and/or tenth aspects of the invention may further comprise introducing the catheter by its proximal end into the urethra (preferably after carrying out the method of the ninth aspect and/or before carrying out the method of the tenth aspect). The method may comprise allowing fluid to pass from the body via the catheter. Consequently, the method may be a method of using a catheter.

According to an eleventh aspect of the invention there is provided a method of unpacking a catheter comprising opening a packaged catheter assembly comprising a catheter arranged within a sterile sealed pouch; the pouch having an openable edge (e.g. an openable top) and a resealing arrangement (which may be proximal the openable edge/top) for resealing the pouch after use; wherein a portion of the catheter is arranged in the sealed pouch so as to obstruct the resealing arrangement; the method comprising opening the openable edge and pulling the catheter out of the sealed pouch.

The method of the eleventh aspect of the invention may further comprise introducing the catheter by its proximal end into the urethra. The method may comprise allowing fluid to pass from the body via the catheter. Consequently, the method may be a method of using a catheter. The method may comprise re-introducing the catheter to the pouch after use, then sealing the pouch with the resealing arrangement. For example, the method may comprise bringing the walls of the pouch together to close the pouch. The method may comprise closing/re-sealing the pouch with the catheter within it. The steps of closing/re-sealing the pouch may be performed after use of the catheter. Thus, the pouch can be used to cleanly store the catheter after use and prior to disposal.

Of course, the packaged catheter assembly may be according to the second aspect of the invention and may include any optional feature outlined above.

According to a twelfth aspect of the invention there is provided a method of wetting a catheter provided in a packaged catheter assembly comprising the catheter and a container of wetting fluid arranged within a pouch; the pouch comprising first and second walls joined around the periphery thereof to define a pair of major edges, a base and a top edge; the pouch having an openable top, which when opened has a mouth at its upper end to allow the catheter to be withdrawn; wherein a lateral join is provided, extending inwardly from a major edge, obscuring visibility of and restricting access to the container of wetting fluid; the method comprising opening the container of wetting fluid in-situ in the pouch, then withdrawing the catheter.

The method of the twelfth aspect of the invention may further comprise introducing the catheter by its proximal end into the urethra. The method may comprise allowing fluid to pass from the body via the catheter. Consequently, the method may be a method of using a catheter.

Of course, the packaged catheter assembly may be according to the third aspect of the invention and may include any optional feature outlined above.

According to a thirteenth aspect of the invention there is provided a method of wetting a catheter provided in a packaged catheter assembly comprising the catheter and a container of wetting fluid arranged within a pouch; the method comprising grasping the pouch at a first interaction region defining a point of contact for a user to grasp a tear away region; and grasping the remainder of the pouch at a corresponding second interaction region defining a point of contact for a user to grasp the remainder of the pouch; the first and second interaction regions comprising one or more visual and/or tactile indicators; the method further comprising opening the container of wetting fluid, the container being arranged at the second interaction region.

Of course, the packaged catheter assembly may be according to the fourth aspect of the invention (and optionally also according to the third aspect of the invention—and indeed any other aspect) and may include any optional feature outlined above.

The method may comprise opening the pouch by tearing it open at the tear away region. The method may comprise opening the container then opening the pouch. The method may comprise simultaneously opening the container and opening the pouch.

The method of the thirteenth aspect of the invention may further comprise introducing the catheter by its proximal end into the urethra. The method may comprise allowing fluid to pass from the body via the catheter. Consequently, the method may be a method of using a catheter.

In any of the methods of wetting the catheter the method may comprise handling the catheter using a handling element, which may comprise a handling tube arranged around part of the catheter tube.

Likewise in any method the method may comprise forming an opening in the pouch, for example opening the pouch along a line of weakness to provide an open mouth.

In any aspect, the catheter is preferably an open catheter, that is that the catheter tube is not coupled to a fluid collection bag, but rather simply directs fluid out of a distal opening (e.g. via a funnel) to a lavatory, urinal or other suitable destination. Of course, embodiments of the invention may also be closed catheter assemblies in which fluid is collected or retained in a fluid collection bag coupled to the catheter.

### DETAILED DESCRIPTION OF THE INVENTION

In order that the invention may be more clearly understood one or more embodiments thereof will now be described, by way of example only, with reference to the accompanying drawings, of which:

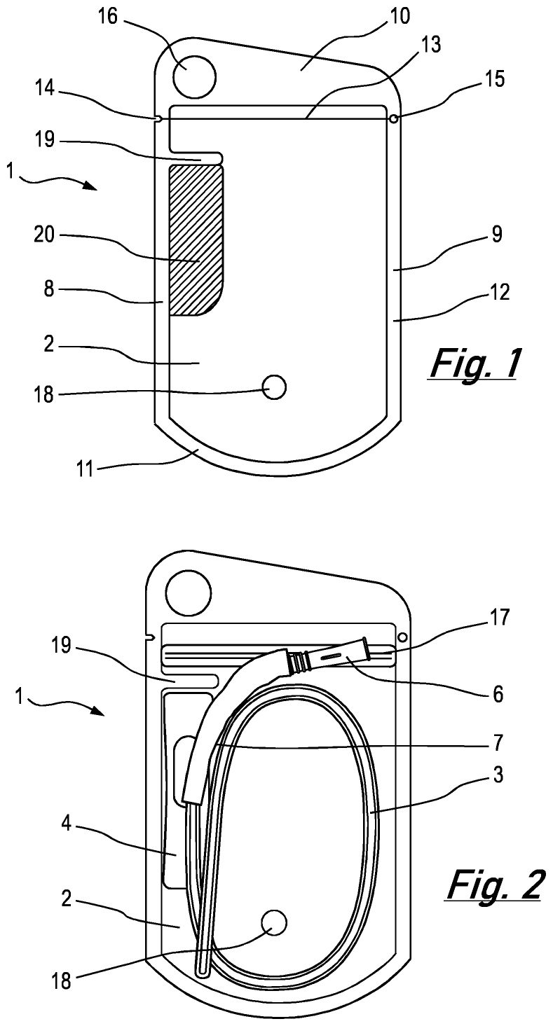

FIG. 1 is a front view of a first embodiment of a packaged catheter assembly;

FIG. 2 is a front view of the packaged catheter assembly of FIG. 1 with internal components visible;

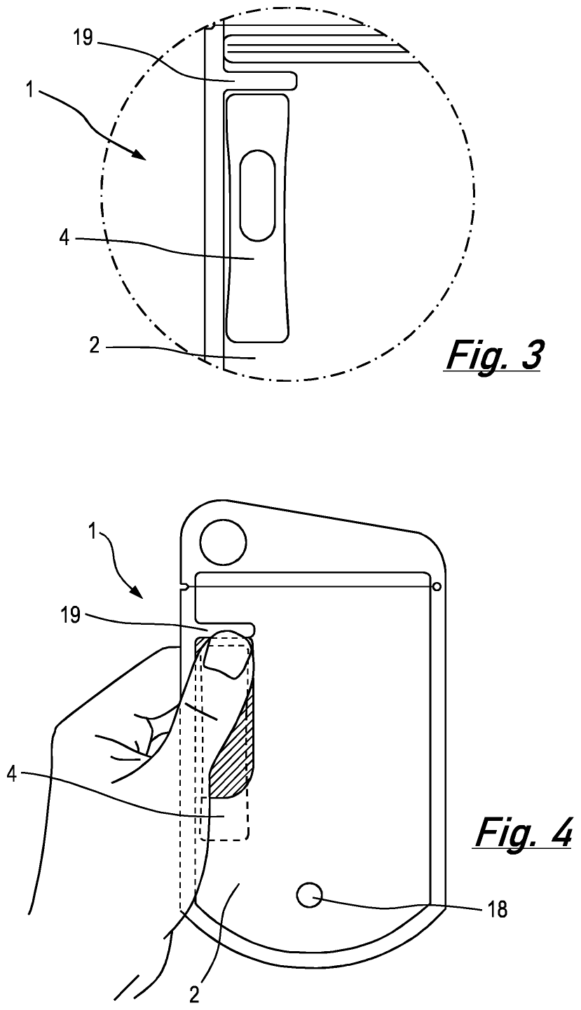

FIG. 3 is an enlarged front view of part of the packaged catheter assembly of FIG. 1 showing the location of the lateral weld and the container of wetting fluid;

FIG. 4 is a front view of the packaged catheter assembly of FIG. 1 showing a user's hand grasping the pouch to open the container of wetting fluid;

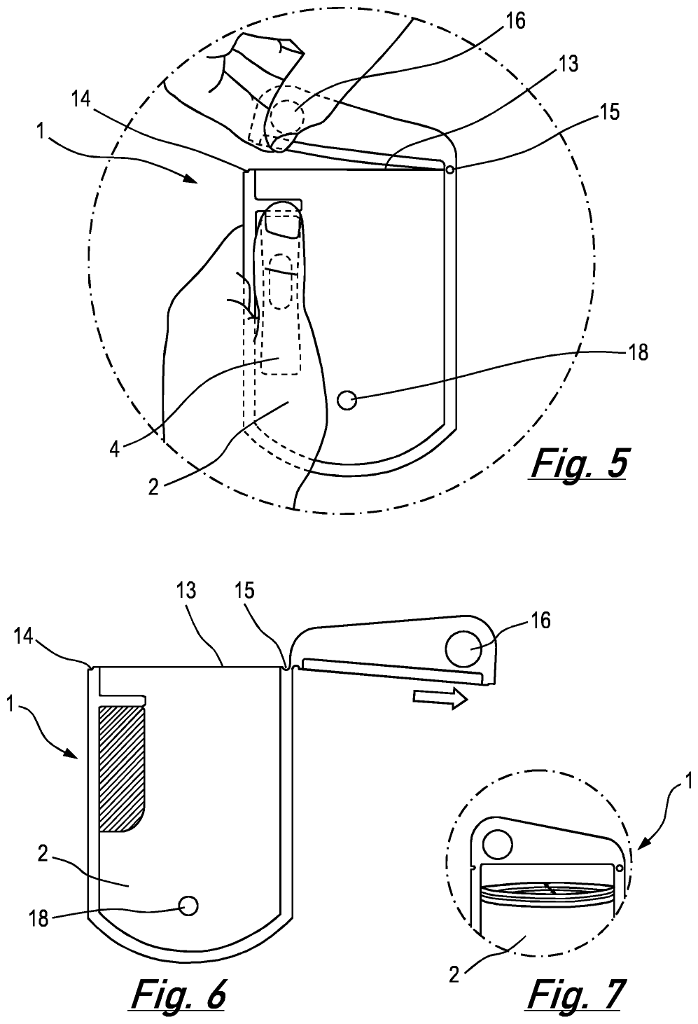

FIG. 5 is a front view the packaged catheter assembly of FIG. 1 showing a user opening the pouch;

FIG. 6 is a front view the packaged catheter assembly of FIG. 1 where top has been fully opened;

FIG. 7 is a front view of the packaged catheter assembly of FIG. 1 showing the resealing arrangement in its open configuration;

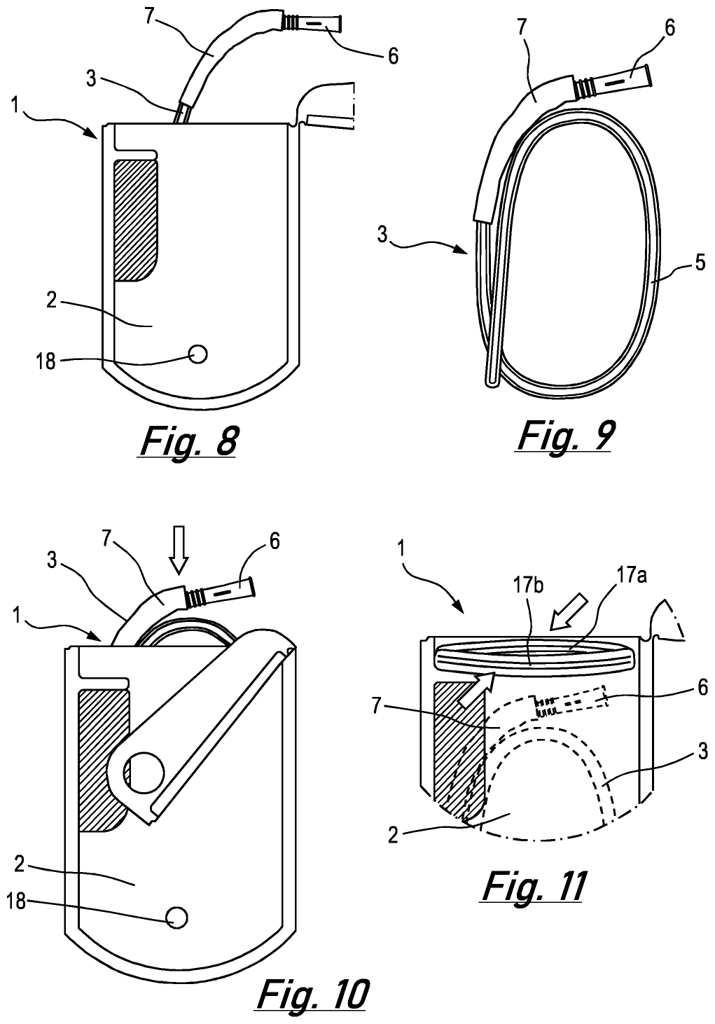

FIG. 8 is a front view showing the catheter assembly being removed from the pouch of FIG. 1;

FIG. 9 is a front view of the catheter assembly of FIG. 1, outside the pouch;

FIG. 10 is a front view of the catheter assembly of FIG. 1 where the catheter is being re-introduced into the pouch;

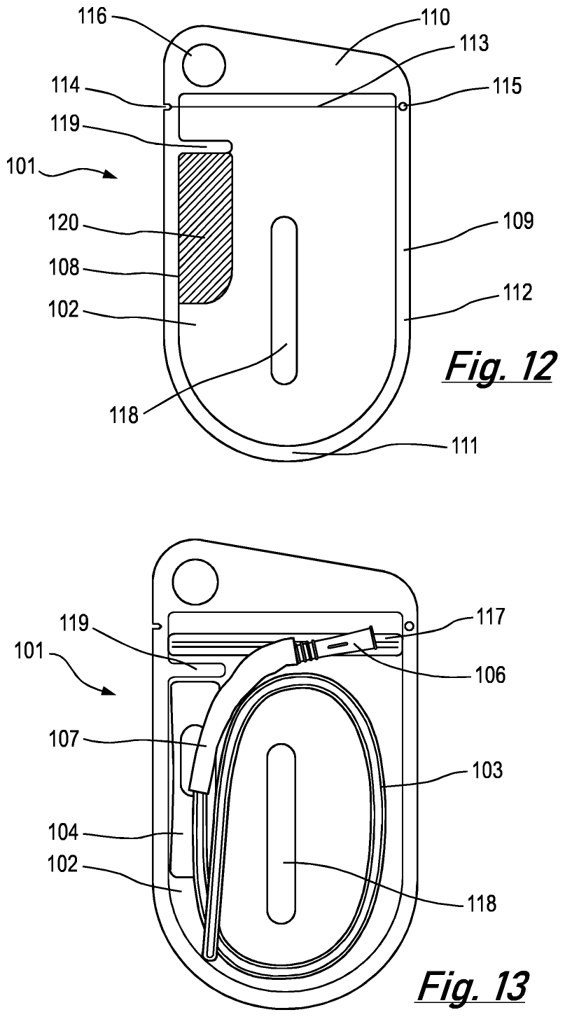

FIG. 11 is a front view of the catheter assembly of FIG. 1 where the catheter has been re-introduced into the pouch and the pouch is ready to be resealed;

FIG. 12 is a front view of a second embodiment of a packaged catheter assembly; and

FIG. 13 is a front view of the packaged catheter assembly of FIG. 12 with internal components visible.

Referring to FIGS. 1-11, a first embodiment of a packaged catheter assembly 1 is shown.

The packaged catheter assembly 1 comprises a pouch 2, which houses a catheter assembly (also referred to as a catheter) 3, best seen in FIG. 9, and a container of wetting fluid 4, best seen in FIGS. 2, 3 and 5.

In this particular embodiment, the catheter 3 is a male intermittent urinary catheter assembly (i.e. sized for use by men and longer than a typical female catheter assembly, although the invention is applicable to female catheter assemblies too and potentially has applicability not only for intermittent urinary catheters). Referring to FIG. 9, the catheter 3 of the embodiment comprises a catheter tube 5, with apertures (not shown) at the proximal end to allow fluid (e.g. urine) to pass from the bladder into the catheter tube 5. The catheter tube of this embodiment is 37 cm long, but of course those skilled in the art will be able to conceive of alternative lengths.

At the distal end of the catheter tube, a funnel 6 is provided to aid the user in directing urine to a suitable location (e.g. a lavatory as the catheter assembly is “open”, not “closed”). Moreover, a gripping element 7 in the form of a handling tube extends around part of the catheter tube 5 and is moveable along the length of the catheter tube 5 to aid in sanitary handling of the catheter 3. Prior to use, the handling tube 7 is arranged adjacent to the funnel 6. The handling tube 7 is short compared to the length of the catheter tube 5, less than a fifth of its length, such that the large majority of the catheter tube 5 is exposed and will therefore be easily wetted prior to use.

In this embodiment, the catheter tube 5 is made from a hydrophilic thermoplastic elastomer (TPE). The handling tube 7 of this example is formed of LDPE. Obviously those skilled in the art will be able to select suitable alternative examples.

In this embodiment, the wetting fluid is water and interacts with the hydrophilic surface of the catheter 3 to render it lubricious. In other embodiments, other wetting fluids may be used and they may be polar (e.g. water-based) or non-polar (e.g. oil-based) depending on the catheter's surface properties.

The container of wetting fluid 4 is a sachet, namely an elongate, rectangular rupturable/burstable sachet. In this embodiment it has a length of 7.8 cm, a width of 2 cm and holds a volume of 6 ml of water. This is a relatively small amount of water for wetting a male catheter assembly, but is adequate because of the unique features of the pouch.

Turning now to the pouch 2 itself, the pouch 2 is formed from two opposing walls of flexible sheet material of identical shape and size which are joined by a peripheral join 12. The flexible sheet material may for example be polypropylene (PP), polyethylene terephthalate (PET), low density polyethylene (LDPE), metalized polyester (MET PET) or orientated polypropylene (OPP); the material may be opaque with a textured or printed appearance and/or feel, such as a matte finish. The pouch 2 may be a different colour from the other components of the packaged catheter assembly, such as any one or more of: the container of wetting fluid 4 and the catheter 3. For example, it may be a dark colour and, these components may be a light colour and thus easily identifiable in the pouch.

The peripheral join 12 is preferably a weld, but in alternative embodiments may be an adhesive join; mechanical seal; heat seal; pressure seal; solvent bond; ultraviolet bond; ultrasonic weld; laser weld; impulse weld; or friction weld. The two walls of flexible sheet material may also be joined by a fold, that is to say, one sheet of flexible material may be folded to provide two opposing walls, which may then be joined to define the remainder of the periphery of the pouch 2.

The peripheral join 12 extends around the entire periphery of the pouch and defines both the outer outline of the pouch (being the outermost outline of the weld) and the inner outline of the pouch (being the innermost outline of the weld). The peripheral join 12 constitutes a sterile seal, thus maintains the sterility of the contents of the pouch 2 (which may of course be sterilised by any approach known in the art) until the seal is broken.

The peripheral join 12 of the pouch 2 defines a pair of lateral, major edges, namely a left-hand edge 8 and a right hand edge 9. In this embodiment the lateral, major edges 8, 9 are parallel and straight, although that is not essential. The pouch 2 also has a top, 10 and an opposing base 11 defined by the peripheral join 12. The top edge of the closed pouch is slanted, being higher at the left hand side than the right hand side, but of course those skilled in the art will be able to conceive of alternatives.

Most notable in terms of one of the aspects of the invention is that the base 11 is contoured and protrudes downwards beyond the point where it meets the lateral edges 8, 9. In particular, in this particular embodiment, the base 11 is rounded, with a part-circular shape.

In this embodiment, the peripheral join is of uniform thickness around the lateral edges 8, 9 and the base 11 of the pouch 2. As such, the shapes of the interior and the exterior of the pouch correspond in these regions. By contrast, at the top of the pouch, the peripheral join is of non-uniform thickness, being broadly trapezoidal, so as to define a sloped exterior upper edge, but a flat interior edge, at a right-angle to the lateral, major, edges 8, 9.

As noted above the protruding base may make the pouch easier to pocket (i.e. to introduce into a pocket, such as a trouser-pocket), and when the pouch is held upright, after opening (e.g. rupturing) the container of wetting fluid 4 (which will normally be done with the pouch upright and closed) the fluid will preferentially accumulate in the protruding base, and on account of the protrusion, a deeper pool of fluid may be provided than would be the case if the base ran straight between the point where it meets the lateral edges. As such, less fluid may be required to provide a sufficient depth to adequately wet the catheter 3. Moreover, because the interior of the base 11 is rounded, when, after use, the used catheter 3 is re-introduced into the pouch 2 prior to disposal, as the tip of the catheter 3 abuts the rounded base 11, it will follow the contour of the base and curl up within the pouch 2.

In another embodiment, not illustrated, the base 11 may be sloped, for example the base may be sloped on two sides, for example, the base 11 may be “triangular”, with two sloped sides extending from the point where the base 11 meets the lateral edges and meeting at a corner, and a third side being defined as the line between the points where the base meets the lateral edges.

In yet another non-illustrated embodiment the base 11 is a combination of rounded and sloped features: in one such example, the base 11 could have two sloped sides, and as such be generally triangular, but could be rounded where the two sloped sides meet, rather than having an abrupt corner; in another such example the base 11 could have two sloped sides, and as such be generally triangular, but could be rounded where the one or more of the sloped sides meet the lateral edges, rather than having an abrupt corner; in yet another such example, the base 11 could have one sloped side and one rounded side, for example, it could comprise a segment of a circle, such as a quarter-circle, extending from one lateral edge, which meets a sloped side, which extends to the other lateral edge.

The top edge of the pouch 2 is openable, in order to form an open mouth through which the catheter 3 can be removed. In this embodiment to open the top, the pouch has a tear away region proximal to an upper edge of the pouch 2. The tear away region, best seen in FIGS. 1, 5 and 6 comprises a tear line 13, which is a line of weakness, e.g. laser-etched, between a first end 14 and a second end 15 of the tear-away region defining a line along which the pouch may be torn to at least partially separate the tear-away region from the remainder of the pouch. The tear line extends just beneath the peripheral seal at the top of the pouch 2, i.e. in a region where the walls of the pouch are not welded to one another. In consequence, tearing here opens the mouth of the pouch 2. The first end 14 is a tear-start, and in this embodiment the tear start takes the form of a triangular cutout extending from the periphery of the pouch into the peripheral weld, so as to preferentially tear through the remainder of the weld along the tear line 13. The second end 15 of the tear away region is a tear-stop, and in this embodiment that takes the form of a circular perforation in the peripheral weld at the opposite end of the tear line. The tear stop, as the name suggests, causes the tearing to stop, meaning that the tear-away region does not entirely tear off from the remainder of the pouch, having formed an open upper end of the pouch 2 in use.

The tear-away region comprises a first interaction region 16, which defines a point of contact for a user to grasp the tear away region. In this embodiment the first interaction region is a visual and tactile indicator, taking the form of an aperture through the generally trapezoidal upper weld towards the left hand side, which in this embodiment is the side where the weld is biggest and (more importantly) corresponds to the side where the tear-start is arranged. The aperture, or “gripping hole” in this embodiment is circular and sized to receive a finger and thumb, being about 1 cm in diameter. Of course, however, the gripping hole may be any suitable size or shape, for example, rectangular, square, circular, elliptical.

Proximal the openable top of the pouch 2, in particular, just beneath the tear line 13, and parallel thereto, at the mouth of the pouch 2, when opened, is a resealing arrangement 17, best seen in FIGS. 2 and 11. The resealing arrangement 17 is intended for resealing the pouch 2 after use and one aspect of the invention concerns holding the resealing arrangement 17 open until the pouch is opened and the catheter 3 is removed. This is achieved by arranging the catheter 3 within the sterile sealed pouch such that a portion of the catheter 3 obstructs the resealing arrangement 17, as will be discussed in more detail below.

The resealing arrangement 17 in this embodiment is made up of two opposing resealing elements, namely opposing resealing strips 17a, 17b, which engage to reseal the pouch 2. The opposing resealing strips 17a, 17b are provided one on each opposing wall of the pouch 2 and span approximately the entire width of the mouth, between the peripheral weld 12. The resealing strips 17a, 17b comprise complementary attachment features on their opposing surfaces which interlock to reseal. In this particular embodiment, the complementary attachment features are a tongue on one resealing strip and corresponding groove on the opposing resealing strip, of the sort commonly known by the Ziploc™ brand. Of course, alternatives could be used, such as strips of hook and loop (as commonly known by the Velcro™ brand), hook and hook, or “mushroom” fastening elements. In other embodiments the resealing arrangement may rely on a single resealing element, such as a single resealing strip, e.g. a strip of adhesive arranged adjacent the openable edge on one wall in order to reseal it by engagement with an opposing wall.

In this embodiment, to obstruct the resealing arrangement 17, a portion of the catheter 3 is arranged between the opposing resealing strips 17a, 17b, to obstruct the resealing arrangement. Specifically, the portion of the catheter that obstructs the resealing arrangement 17 includes the funnel 6, which lies at an oblique angle (approximately 20 degrees) across the resealing strips 17a, 17b, physically separating them from one another. The funnel 6 extends across at least 50% of the height of the resealing strips, namely extending across 100% of the height of the resealing strips. In addition to the funnel 6 being arranged in register with and physically between the resealing elements, the portion of the catheter 3 that obstructs the resealing arrangement 17 also includes the distal end of the catheter tube 3 where it joins to the funnel 6, which is also arranged physically between the resealing strips 17a, 17b and a small section of the handling tube 7 which is also arranged between the resealing strips 17a, 17b. As can be seen well in FIG. 2, the catheter is arranged in a curled up arrangement, extending from the funnel at the top right hand side of the pouch, across towards the left hand side, then extending down to the bottom of the pouch, back from left to right following the base 11 of the pouch, then up the right hand side, back across just under the funnel 6 and handling tube 7 and down the left hand side. The catheter 3 maintains this shape whilst the pouch is sealed closed, so the resealing arrangement remains obstructed. The catheter 3 is formed of a resilient material, and as such it is biased towards a straighter shape, and is therefore urged against the base 11 of the pouch and towards the top of the pouch. A secondary weld 18 is provided joining the walls of the pouch 2 near its base 11, defining a channel with the base, through which the catheter tube 3 extends. The secondary weld 18 acts to ensure that as the catheter 3 is pulled out by the funnel, it unwinds and all areas of the catheter tube between the secondary weld 18 and the proximal end are forced through the channel, near the base 11, where the wetting fluid will pool.

In light of the shallow angle at which the funnel 6 and portions of the catheter tube 3 and handling tube 7 extend across the resealing arrangement 17, they physically separate about 50% of the strips from one another, as such, that 50% cannot be sealed before use. Moreover, to either lateral side of the portion of the catheter 3 that blocks the resealing arrangement, an air gap is formed and the resealing arrangement is urged apart by its natural resilience.

The portion of the catheter 3 obstructs the resealing arrangement 17 such that not only is it is not fully closed before use, but is at least 60% unsealed before use.

As can be seen from FIG. 2 the top of the tip of the funnel 6 is not in register with the resealing strips 17a, 17b, but extends beyond the resealing arrangement 17 into a region between the openable edge and the resealing arrangement 17. Indeed, the catheter extends over at least 80% and at least 90% of the height of the interior of the pouch, namely about 95% of the height of the pouch, and extends over at least 80% and at least 90% of the distance from the base of the interior of the pouch to the top of the resealing arrangement, namely over about 95% of the distance from the base of the pouch to the top of the resealing arrangement. The majority of the catheter assembly 2 and the container of wetting fluid on the other hand are located in the body of the pouch 2, beneath the resealing arrangement 17.

Just under the resealing arrangement 17, extending inwardly from the peripheral join 12 at one major edge, namely the left lateral edge 8 in this particular embodiment, a lateral join 19 is provided. The lateral join 19 is carefully arranged with respect to the container of wetting fluid 4 in order to obscure visibility of and access to the container of wetting fluid 4. In particular, the container of wetting fluid 4 is arranged adjacent the major edge 8 from which the lateral weld 19 extends, between the lateral join 19 and the base 11 of the pouch 2.

The advantage of this arrangement is that if the container 4 cannot be seen from the top, it is unlikely that a user will remove it. Consequently, it is hoped and expected that it is more likely that a user will open the container of liquid in-situ. The benefit of this is that spillage of the wetting fluid is less likely and therefore more of the fluid will be present in the pouch to fulfil its function of wetting the catheter 3 (and users are less likely to wet their hands, which is undesirable). The lateral join 19 may also act to block fluid from spurting out of the top of the pouch, if it is ruptured after opening the pouch 2 and may reduce the chances of the container itself being accidentally squeezed out of the pouch 2 instead of rupturing.

Conveniently, the lateral join 19 is formed as a weld, in particular a plastic weld between opposing walls of plastics material formed simultaneously with the formation of the peripheral join.

Being arranged just beneath the resealing arrangement 17, the lateral join 19 is proximate to the mouth of the pouch, no more than 5% of the length of the pouch from the mouth. The lateral join 19 defines the length of the opening to access the contents of the pouch; the length of the opening being the distance from the tip of the lateral join 19 (distal the major edge 8 from which it extends) to the opposing major edge 9. Of course, this means that the length of the opening is shorter than the distance between the major edges, which define the length of the mouth, and as such, the lateral join is preferably fairly short, e.g. about 2 cm long.

In this embodiment the lateral join 19 is perpendicular to the major edge 8 from which it extends, but it could extend at other angles.

It will be recalled that a first interaction region 16 is provided at the tear-away region, namely in the join at the top of the pouch 2 towards the left hand side, another inventive aspect concerns the provision of a second interaction region 20. The second interaction region 20 defines a point of contact for a user to grasp the remainder of the pouch 2, below the tear line 13, in order to tear the tear away region away from the remainder of the pouch (with one hand interacting with each interaction region).

The second interaction region 20 again comprises one or more visual and/or tactile indicators, and in this embodiment is provided as a printed pattern, in the form of patterned dots, intended to replicate the appearance of a grip, to encourage gripping. Notably, the second interaction region 20 is provided at the same major edge as the first interaction region 16, and most notably, the container of wetting fluid 4 is arranged at the second interaction region 20, such that squeezing the pouch 2 at the second interaction region 20 ruptures the container of wetting fluid 4.

In this embodiment the second interaction region 20 is shaped to substantially match the shape of the container of wetting fluid 4, being generally rectangular (but having a rounded lower right hand edge).

The purpose of arranging the second interaction region 20 in juxtaposition with the container of wetting fluid 4 is, as outlined above, to encourage the opening of the wetting fluid before the pouch 2 is opened, in order to reduce the prospect of spillage. Thus, the container of wetting fluid 4 is rupturable and is configured to rupture under the squeezing force that will typically be required to grasp the second interaction region 20 tightly enough to open the pouch, in order that the container of wetting fluid 4 is opened when the user grasps the second interaction region 20, just prior to opening, if it has not already been opened.

The second interaction region 20 overlaps substantially with the container of wetting fluid 4. Thus, as can be seen from FIG. 4, for example, the second interaction region extends from the major edge of the towards an opposing major edge, and the container of wetting fluid 4 is located beneath the wall upon which the second interaction region is arranged and in juxtaposition with the second interaction region 20.

The second interaction region 20 may completely overlap the container of wetting fluid 4, i.e. the outline of the container of wetting fluid 4 may be entirely within the outline of the second interaction region 20, however, in this particular embodiment, that is not the case, and the container of fluid extends further towards the base 11 of the pouch 2. Nonetheless, if pressure is applied to the entirety of the second interaction region 20, that pressure is sure to bear on the container of wetting fluid 4. The container of wetting fluid 4 may completely overlap the second interaction region 4, i.e. the outline of the second interaction region 20 may be entirely within the outline of the container of wetting fluid 4. Again, however, in this embodiment that is not the case, as the second interaction region 20 extends slightly beyond the right hand edge of the container of wetting fluid 4, as mentioned above.

Thus, the embodiment is one in which the second interaction region 20 and the container of wetting fluid 4 are partially overlapping, i.e. the second interaction region 20 overlaps only part of the container of wetting fluid 4 and the container of wetting fluid 4 overlaps only part of the second interaction region 20, albeit the overlap is substantial, with at least 50% of the second interaction region overlapping with at least 50% of the container of wetting fluid 4. As such, even if the user does not grasp the entirety of the second interaction region 20, but only say half of it (as shown by the thumb in FIG. 4, sufficient pressure will reach the container of wetting fluid to cause it to rupture and form a pool at the base.

In another embodiment, not shown, the second interaction region 20 comprises a rupture indicator, corresponding to a point which when squeezed will rupture the container of wetting fluid 4. For example, the rupture indicator may be a mark, e.g. a visible mark of a different colour to the remainder of the interaction region 20.

Obviously it is important that the container of wetting fluid 4 remains in position beneath the second interaction region 20. In this embodiment it is simply wedged into position (i.e. gripped by the two walls between which it is arranged). In other embodiments, not shown, it could be maintained in position by arranging the contents of the pouch 2 so as to hold the container of wetting solution 4 in position; for example the catheter 3 could be curled up so as to bear on the side of the container of wetting solution 4 opposing the major edge 8 that it is adjacent to, and thereby press it against the major edge 8, or it could be adhered or welded in position, or held in position by the secondary join 18.

In use, a user will hold the pouch generally upright, with its openable top above its base 11 and grasp the pouch 2 at the second interaction region 20, as shown in FIG. 4, squeezing it and hence rupturing the container of wetting fluid 4, such that the wetting fluid flows freely to the base 11, where it forms a pool. The user then grasps the first interaction region 16, without having to move their hand from the second interaction region 20, or only moving it slightly, and, as shown in FIG. 5, tears open the top, with the tear opening from the tear start at the first end 14 of the tear line 13 and ending at the tear stop 15, thereby opening the mouth of the pouch, but not tearing off the tear away region completely, as can be seen by FIG. 6.

Having opened the container of wetting fluid 4 and the mouth of the pouch 2, the user is presented with the funnel 6 sticking out through the resealable strips 17a, 17b. Notably, the resealable strips will be open (at least partially), so the user will not have to open them for access. Being arranged above the container of wetting fluid 4 and indeed above the lateral join 19, the funnel 6 will be dry, as will much of the handling tube 7. The user will then draw out the catheter 3, holding it by the funnel 6, as shown in FIG. 8. As the catheter 3 is drawn out, the secondary join 18 will bear on the catheter tube, forcing the entirety of the tube that is between the secondary join 18 and its proximal tip to be drawn through the wetting fluid which is pooled in the channel between the secondary join 18 and the base 11 of the pouch 2. Thus, the majority of the catheter 3 will be wetted as it is pulled out, and importantly the proximal part of the catheter 3, which will travel furthest through the urethra, is well wetted.

The user can then insert the catheter 3 into the urethra and drain the bladder as usual, using the handling tube 7 to aid insertion into the urethra. Once the bladder has been adequately drained, the user can re-introduce the catheter to the pouch 2 to store it prior to disposal. The user can introduce the catheter 3 by its proximal tip, and push it down alongside one major edge 8/9 until it reaches the base 11. The base 11, being contoured, will serve to direct the catheter across the base and back up the opposite side, to aid curling it back up in the pouch 2. Alternatively, a user could curl the catheter 3 up, back into its pre-use configuration, and insert the entire curled up catheter assembly back into the pouch, as shown in FIG. 10, then, once it is pushed down beneath the resealing arrangement 17, the resealing strips 17a, 17b, can be squeezed against one another to reseal the (no longer sterile) catheter back in the pouch ready for disposal.

Referring to FIGS. 12 and 13, a second embodiment of a packaged catheter assembly 101 is shown. The second embodiment shares many of the features of the first embodiment described above, and so only differences in the features are described below, and like numerals, incremented by 100 are used to denote like features unless described differently below.

In effect there are only two differences: the shape of the base 111 and the shape of the secondary weld 118.

Whereas the peripheral weld 11 of the first embodiment is part-circular, the base 111 of the second embodiment is semi-circular. The semi-circular base extends smoothly from the straight lateral sides 108, 109, and the peripheral seal 112 is once again of uniform thickness in at the lateral sides and base, so both the exterior and interior are contoured in the same fashion, namely as a semi-circle. This arrangement is particularly effective in terms of aiding in curling up the catheter 103 when re-introducing it to the pouch 102, and of course is also useful in terms of easy pocketing of the packaged catheter assembly 101.

The secondary weld 118 of the second embodiment is elongate and extends lengthways along at the centre of the pouch 102. In this embodiment, the shape of the secondary weld 118 is that of a (long) stadium, that is to say, a rectangle with semicircles at its opposite sides (upper and lower), but of course that is a matter of design and other elongate shapes would also work. The base of the elongate secondary weld 118 is roughly aligned with the point at which the semi-circular base 111 meets the lateral sides 108, 109 and it extends upwards beyond half-way up the height of the pouch 2, alongside a substantial portion of the container of wetting fluid 104, alongside about 60% of the container of wetting fluid. As such, the pouch is “pinched” along its centreline by the weld. This inhibits movement of the catheter 103, in particular lateral movement of the catheter 103 where it is alongside/overlying the container of wetting fluid 104, improving the extent to which it is wedged into position.

In terms of use, the approach is very similar to the first embodiment: a user will hold the pouch 102 generally upright, with its openable top above its base 111 and grasp the pouch 102 at the second interaction region 120, squeezing it and hence rupturing the container of wetting fluid 104, such that the wetting fluid flows freely to the base 111, where it forms a pool. The user then grasps the first interaction region 116, without having to move their hand from the second interaction region 120, or only moving it slightly, and tears open the top, with the tear opening from the tear start at the first end 114 of the tear line 113 and ending at the tear stop 115, thereby opening the mouth of the pouch 102, but not tearing off the tear away region completely.

Having opened the container of wetting fluid 104 and the mouth of the pouch 102, the user is presented with the funnel 106 sticking out through the resealing arrangement 117. The user will then draw out the catheter 103, holding it by the funnel 106. As the catheter 103 is drawn out, the bottom of the secondary join 118 will bear on the catheter tube, forcing the entirety of the tube that is between the secondary join 118 and its proximal tip to be drawn through the wetting fluid which is pooled in the channel between the secondary join 118 and the base 111 of the pouch 102, wetting the catheter 103 well.

The user can then insert the catheter 103 into the urethra and drain the bladder as usual, using the handling tube 107 to aid insertion into the urethra. Once the bladder has been adequately drained, the user can re-introduce the catheter to the pouch 102 to store it prior to disposal. Notably, whereas in the first embodiment it would be possible for a user to curl the catheter up into its pre-use configuration and introduce the whole thing into the pouch, the elongate weld 118 inhibits this approach in the second embodiment. As such, the user will introduce the catheter 103 by its proximal tip, and push it down alongside one major edge 108/109 until it reaches the base 111. The base 111, being semi-circular, easily directs the catheter across the base and back up the opposite side, to aid curling it back up in the pouch 102. Of course, once it is arranged entirely beneath the resealing arrangement 117, the resealing elements, can be squeezed against one another to reseal the (no longer sterile) catheter back in the pouch ready for disposal.

The one or more embodiments are described above by way of example only. Many variations are possible without departing from the scope of protection afforded by the appended claims.

1. A packaged catheter assembly comprising a catheter arranged within a sterile sealed pouch; the pouch having an openable top and a resealing arrangement proximal the openable top for resealing the pouch after use; wherein a portion of the catheter is arranged in the sealed pouch so as to obstruct the resealing arrangement.

2. A packaged catheter assembly according to claim 1 wherein the resealing arrangement comprises opposing resealing elements.

3. A packaged catheter assembly according to claim 2 wherein the opposing resealing elements are opposing strips, which engage to reseal the pouch.

4. A packaged catheter assembly according to claim 2 or 3 wherein the portion of catheter is arranged between the opposing resealing elements to obstruct the resealing arrangement.

5. A packaged catheter assembly according to claim 2 or 3 wherein the portion of the catheter is arranged adjacent to the opposing resealing elements to obstruct the resealing arrangement.

6. A packaged catheter assembly according to any of claims 2 to 5 wherein the opposing resealing elements comprise complementary attachment features on their opposing surfaces which interlock to reseal; preferably wherein the opposing resealing elements are opposing resealing strips having a tongue and groove arrangement.

7. A packaged catheter assembly according to any preceding claim wherein the portion of the catheter extends across at least 50% of the height of the resealing arrangement.

8. A packaged catheter assembly according to claim 1 wherein the resealing arrangement comprises a strip of adhesive arranged adjacent the openable edge on one wall in order to reseal it by engagement with an opposing wall; preferably wherein the portion of the catheter is arranged between the strip of adhesive and the region of the opposing wall that opposes the strip of adhesive.

9. A packaged catheter assembly according to any preceding claim wherein the portion of the catheter extends over at least 90% of the height of the interior of the pouch, and/or at least 90% of the distance from the base of the interior of the pouch to the top of the resealing arrangement.

10. A packaged catheter assembly according to any preceding claim wherein the portion of the catheter obstructs the resealing arrangement such that it is not fully closed before use; preferably wherein the portion of the catheter obstructs the resealing arrangement such that it is at least at least 50%, unsealed before use.

11. A packaged catheter assembly according to any preceding claim wherein the catheter is formed of a resilient material, and urged against the base of the pouch and towards the top of the pouch.

12. A packaged catheter assembly according to any preceding claim wherein the portion of the catheter extends into a region between the openable edge and the resealing arrangement.

13. A packaged catheter assembly according to claim 12 wherein the pouch has a construction comprising a body, in which the majority of the catheter assembly, and optionally a container of wetting fluid is located, the resealing arrangement and the region between the openable edge and the resealing arrangement, with the portion of the catheter which obstructs closure of the resealing arrangement before use extending all the way to the region between the resealing arrangement and the openable edge.

14. A packaged catheter assembly according to any preceding claim wherein the portion of catheter that is arranged to obstruct the resealing arrangement is the distal end of the catheter.

15. A packaged catheter assembly according to claim 14 wherein the portion of the catheter that is arranged to obstruct the resealing arrangement is a funnel.

16. A packaged catheter assembly according to any preceding claim wherein the portion of catheter that is arranged to obstruct the resealing arrangement is a gripping element.

17. A packaged catheter assembly according to claim 16 wherein the gripping element is a handling tube extending around part of the catheter tube and moveable along the length of the catheter tube.

18. A packaged catheter assembly according to any preceding claim wherein the catheter is a closed-catheter assembly, with a catheter tube attached to a fluid collection bag, and wherein the fluid collection bag is arranged to obstruct the resealing arrangement.

19. A packaged catheter assembly according to any preceding claim further comprising a tear-away region at the openable edge of the pouch, wherein the tear away region is configured to open the openable edge and thereby break the sterile seal.

20. A packaged catheter assembly according to any preceding claim wherein a lateral join is provided, extending inwardly from a major edge, obscuring visibility of and restricting access to the container of wetting fluid.

21. A packaged catheter assembly according to any preceding claim wherein the pouch has a tear-away region defined proximal to an upper edge of the pouch, the tear away region comprising a tear line between a first end and a second end of the tear-away region defining a line along which the pouch may be torn to at least partially separate the tear-away region from the remainder of the pouch to form an open upper end of the pouch, in use; the tear-away region comprising a first interaction region defining a point of contact for a user to grasp the tear away region; and the remainder of the pouch having a corresponding second interaction region defining a point of contact for a user to grasp the remainder of the pouch; the first and second interaction regions comprising one or more visual and/or tactile indicators, being provided at the same major edge and being separated from each other by the tear line; and the container of wetting fluid being arranged at the second interaction.

22. A packaged catheter assembly according to any preceding claim, the pouch having a pair of lateral edges, a top and an opposing base; wherein the base protrudes downwards beyond the point where it meets the lateral edges.

23. A method of forming a packaged catheter assembly comprising a catheter arranged within a sterile sealed pouch; method comprising forming a pouch having an openable top; providing a resealing arrangement proximal the openable top for resealing the pouch after use; arranging a portion of the catheter so as to obstruct the resealing arrangement when the pouch is sealed with the sterile seal; and sealing and sterilising the pouch.

24. A method according to claim 23, wherein the packaged catheter assembly is a packaged catheter assembly according to any of claims 1 to 22.
